(19) United States
(12) Patent Application Publication
(10) Pub. No.: US 2014/0099275 A1
(43) Pub. Date: Apr. 10, 2014

(54) LIGHTWEIGHT STYLING AGENTS

(71) Applicant: Henkel AG & Co. KGaA, Duesseldorf (DE)

(72) Inventors: Thorsten Knappe, Schenefeld (DE); Julia Bibiane Lange, Sievershuetten (DE)

(73) Assignee: Henkel AG & Co. KGaA, Duesseldorf (DE)

(21) Appl. No.: 14/096,233

(22) Filed: Dec. 4, 2013

Related U.S. Application Data
(63) Continuation of application No. PCT/EP2012/060131, filed on May 30, 2012.

(30) Foreign Application Priority Data
Jun. 8, 2011 (DE) 10 2011 077 233.2

(51) Int. Cl. A61K 8/81; A61Q 5/06; A61K 8/92

(52) U.S. Cl. CPC A61K 8/8182; A61K 8/92; A61K 8/925; A61K 8/8152; A61Q 5/06
USPC 424/70.15

(57) ABSTRACT

A styling agent in a cosmetically acceptable carrier includes, based in each case on its weight: 0.01 to 3 wt % of at least one copolymer A as defined herein, and 0.05 to 10 wt %, by preference 0.1 to 7.5 wt %, more preferably 0.25 to 6 wt %, and in particular 0.5 to 5 wt % of at least one film-forming polymer, and 0.5 to 30 wt %, by preference 1 to 25 wt %, more preferably 2.5 to 20 wt %, and in particular 5 to 15 wt % of at least one wax having a melting point in a range from 40° C. to 90° C. The agent has a high degree of hold and a pleasant care-providing feel, and is not heavy.

### FIELD OF THE INVENTION

The present invention generally relates to agents for the temporary deformation of keratinic fibers, containing a combination of at least one special polymer with further special ingredients; to the use of said agents for the temporary deformation of keratinic fibers; and to a corresponding method.

### BACKGROUND OF THE INVENTION

“Keratinic fibers” are understood in principle as all animal hair, e.g. wool, horsehair, angora wool, furs, feathers, and products or textiles produced therefrom. Preferably, however, the keratinic fibers are human hairs.

An attractive-looking hairstyle is generally regarded these days as an indispensable element of a well-groomed appearance. Given the currents of fashion, more and more hairstyles regarded as chic are ones that, for many types of hair, can be constructed, or maintained for a longer period of time of up to several days, only with the use of setting active substances. Hair treatment agents that serve for permanent or temporary shaping of the hair therefore play an important role. Temporary shaping results that are intended to result in good hold without impairing the healthy appearance of the hair, for example its shine, can be achieved e.g. using hair sprays, hair waxes, hair gels, hair foams, blow-dry waves, etc.

Corresponding agents for temporary shaping usually contain synthetic polymers as a shaping component. Preparations that contain a dissolved or dispersed polymer can be applied onto the hair by means of propellant gases or using a pump mechanism. Hair gels and hair waxes in particular, however, are as a rule not applied directly onto the hair but instead distributed in the hair using a comb or one's hands.

The most important property of an agent for the temporary deformation of keratinic fibers, hereinafter also called a “styling agent,” is to impart the strongest possible hold to the treated fibers in the shape that is generated. If the keratinic fibers involved are human hairs, terms also used are a strong “hairstyle hold” or a high “degree of hold” of the styling agent. The hairstyle hold is determined substantially by the nature and quantity of the setting active substances used, although the further constituents of the styling agent can also have an influence.

One problem is the fact that the product consistency of such products is assessed negatively by the consumer, in that these products are regarded as viscous, sticky, and difficult to apply, and leave behind an oily, heavy feel. In addition, consumers want products that do not weigh down the hair and do not produce “helmet hair”; along with good hold, the hairstyle should be remodulatable, and the hair's shine should not be impaired.

The object of the present invention was therefore to make available an agent for the temporary deformation of keratinic fibers that is notable for a very good degree of hold without needing to sacrifice remodulability, a well-groomed hair feel and shine, and pleasant product haptics.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A cosmetic agent containing in a cosmetically acceptable carrier, based in each case on its weight: 0.01 to 3 wt % of at least one copolymer A that contains at least one structural unit according to formula (I) and at least one structural unit according to formula (II)

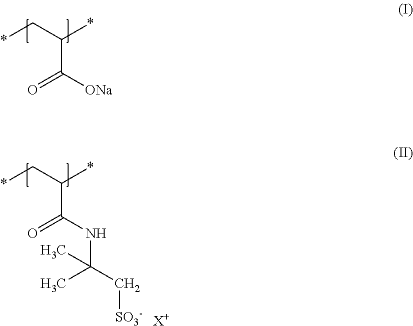

in which X⁺ denotes a physiologically acceptable cation; and 0.05 to 10 wt %, by preference 0.1 to 7.5 wt %, more preferably 0.25 to 6 wt %, and in particular 0.5 to 5 wt % of at least one film-forming polymer; 0.5 to 30 wt %, by preference 1 to 25 wt %, more preferably 2.5 to 20 wt %, and in particular 5 to 15 wt % of at least one wax having a melting point in a range from 40° C. to 90° C.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now been found, surprisingly, that non-weighting styling products having a high degree of hold and a pleasant care-providing feel in the hair can be made available by incorporating a combination of special polymers with waxes into the agent.

A first subject of the present invention is therefore a cosmetic agent containing in a cosmetically acceptable carrier, based in each case on its weight:
a) 0.01 to 3 wt % of at least one copolymer A that contains at least one structural unit according to formula (I) and at least one structural unit according to formula (II)

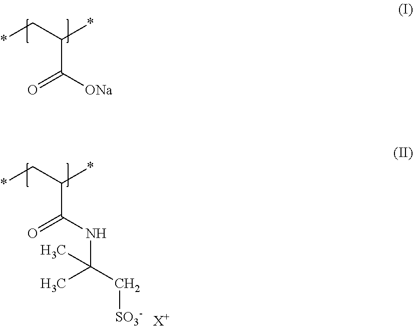

in which X⁺ denotes a physiologically acceptable cation,

and
b) 0.05 to 10 wt %, by preference 0.1 to 7.5 wt %, more preferably 0.25 to 6 wt %, and in particular 0.5 to 5 wt % of at least one film-forming polymer,
c) 0.5 to 30 wt %, by preference 1 to 25 wt %, more preferably 2.5 to 20 wt %, and in particular 5 to 15 wt % of at least one wax having a melting point in a range from 40° C. to 90° C.

The agents according to the present invention contain as a first obligatory component a polymer that is constructed from at least two different monomers of formulas (I) and (II). Further monomers can additionally be polymerized in.

The first monomer that is contained in copolymer A is sodium acrylate, i.e. the sodium salt of acrylic acid. In addition thereto, acrylic acid can also be present as a further monomer module in the polymers, but this is not absolutely necessary according to the present invention.

The second monomer that is contained in copolymer A is 2-methyl-2[(1-oxo-2-propenyl)amino]-1-propanesulfonic acid (AMPS), which can be present in partly or entirely neutralized form. Na⁺ and NH₄⁺ are usually preferred as cations.

The monomers of formulas (I) and (II) are contained in copolymers A by preference in a quantity and distribution such that copolymer A exhibits molar masses between 5 and 1000 kDa. Preferred agents according to the present invention are characterized in that they contain copolymer(s) A having molar masses from 10 to 750 kDa, by preference from 25 to 500 kDa, more preferably from 50 to 400 kDa, and in particular from 70 to 250 kDa.

The monomers of formulas (I) and (II) are by preference contained in copolymer A within specific limits. Agents preferred according to the present invention are characterized here in that they contain copolymer(s) A that contain
10 to 90 mol %, by preference 15 to 85 mol %, and in particular 20 to 80 mol % monomers of formula (I) and
10 to 90 mol %, by preference 15 to 85 mol %, and in particular 20 to 80 mol % monomers of formula (II).

Regardless of whether the agents according to the present invention contain one or more copolymers A, it is preferred to use copolymers A within specific quantity ranges. Agents according to the present invention that contain, based on the weight of the ready-to-use agent, 0.025 to 2.5 wt %, preferably 0.05 to 2 wt %, and in particular 0.1 to 1 wt % copolymer(s) A are preferred here.

Particularly preferred copolymers A are partly or entirely neutralized, crosslinked copolymers made up of 2-methyl-2[(1-oxo-2-propenyl)amino]-1-propanesulfonic acid (AMPS) and acrylic acid. These are commercially obtainable in the form of an inverse, self-inverting latex, for example under the commercial names Simulgel® EG and Simulgel® EPG of the Seppic company.

Also particularly preferred are partly or entirely neutralized, crosslinked copolymers of 2-methyl-2[(1-oxo-2-propenyl)amino]-1-propanesulfonic acid (AMPS), acrylic acid and/or sodium acrylate, and (as a third monomer module) dimethyl acrylamide, commercially obtainable in the form of an inverse, self-inverting latex e.g. under the commercial name Simulgel® SMS 88 of the Seppic company.

The copolymers A contained in the agents according to the present invention can contain further monomers besides monomers (I) and (II). Copolymers A that contain at least one structural unit according to formula (I) and at least one structural unit according to formula (II) and at least one structural unit according to formula (III)

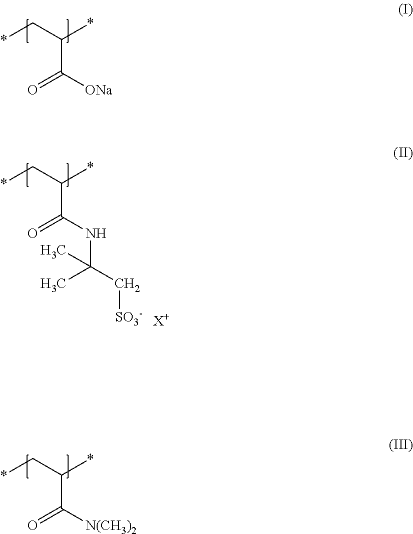

in which X⁺ denotes a physiologically acceptable cation, have proven particularly successful here.

In this embodiment the third monomer that is contained in copolymer A is dimethyl acrylamide. Copolymers A of this preferred embodiment can be described by the general formula

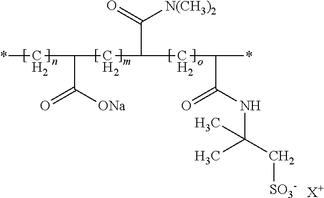

where the indices m and n and o each vary depending on the molar mass of the polymer and are not intended to signify that these are necessarily block copolymers. Structural units of formulas (I), (II), and (III) can instead also be present in statistically distributed fashion in the molecule.

The monomers of formulas (I), (II), and (III) are contained in copolymers A of this preferred embodiment by preference in a quantity and distribution such that copolymer A of this preferred embodiment has molar masses between 5 and 1000 kDa.

The monomers of formulas (I), (II), and (III) are by preference contained in copolymer A within specific limits. Agents according to the present invention of this preferred embodiment are characterized here in that they contain copolymer(s) A that contain
10 to 90 mol %, by preference 15 to 85 mol %, and in particular 20 to 80 mol % monomers of formula (I) and
5 to 85 mol %, by preference 7.5 to 80 mol %, and in particular 10 to 60 mol % monomers of formula (II), and
5 to 85 mol %, by preference 10 to 80 mol %, and in particular 15 to 70 mol % monomers of formula (III).

As a second essential ingredient, the agents according to the present invention contain 0.05 to 10 wt %, by preference 0.1 to 7.5 wt %, more preferably 0.25 to 6 wt %, and in particular 0.5 to 5 wt % of at least one film-forming polymer.

Multiple film-forming and/or setting polymers can of course also be contained. These film-forming and/or setting polymers can be both permanently and also temporarily cationic, anionic, nonionic, or amphoteric. When at least two film-forming and/or setting polymers are used, they can of course have different charges. It can be preferred according to the present invention if an ionic film-forming and/or setting polymer is used together with an amphoteric and/or nonionic film-forming and/or setting polymer. The use of at least two oppositely charged film-forming and/or setting polymers is also preferred. In the latter case, a particular embodiment can in turn additionally contain at least one further amphoteric and/or nonionic film-forming and/or setting polymer.

Because polymers are often multifunctional, their functions cannot always be clearly and unequivocally demarcated from one another. This applies in particular to film-forming and setting polymers. Many polymers that are described primarily as film-forming also have setting properties, and vice versa. It is therefore explicitly noted at this juncture that in the context of the present invention, both film-forming and setting polymers are essential. Because the two properties are also not entirely independent of one another, the term “setting polymers” is also always understood as “film-forming polymers,” and vice versa.

Included among the preferred properties of the film-forming polymers is film formation. “Film-forming polymers” are to be understood as those polymers that, upon drying, leave behind a continuous film on the skin, hair, or nails. Film-formers of this kind can be used in a very wide variety of cosmetic products such as, for example, face masks, make-up, hair setting agents, hair sprays, hair gels, hair waxes, hair therapies, shampoos, or nail polishes. Particularly preferred are those polymers that possess sufficient solubility in water, alcohol, or water/alcohol mixtures. It is thereby possible to produce corresponding solutions that can be used or further processed in simple fashion. The film-forming polymers can be of synthetic or natural origin. “Film-forming polymers” are furthermore understood as those polymers which, when used in a 0.01 to 20 wt % aqueous, alcoholic, or aqueous alcoholic solution, are capable of depositing a transparent polymer film onto the hair. The film-forming polymers can be anionically, amphoterically, nonionically, permanently cationically, or temporarily cationically charged.

Suitable synthetic film-forming, hair-setting polymers that are preferred according to the present invention are homo- or copolymers that are constructed from at least one of the following monomers: vinylpyrrolidone, vinylcaprolactam, vinyl esters such as, for example, vinyl acetate, vinyl alcohol, acrylamide, methacrylamide, C₁ to C₇ alkyl acrylamide, C₁ to C₇ dialkyl acrylamide, C₁ to C₇ alkyl methacrylamide, C₁ to C₇ dialkyl methacrylamide, C₁ to C₇ alkyl acrylate, acrylic acid, propylene glycol, ethylene glycol, where the C₁ to C₇ alkyl groups of these monomers are by preference C₁ to C₃ alkyl groups.

Homopolymers of vinyl caprolactam, of vinylpyrrolidone or of N-vinylformamide may be recited by way of example. Further suitable synthetic film-fanning, hair-setting polymers are, for example, copolymers of vinylpyrrolidone and vinyl acetate, terpolymers of vinylpyrrolidone, vinyl acetate, and vinyl propionate, polyacrylamides that are marketed, for example, under the commercial names Akypomine® P 191 of the CHEM-Y company, Emmerich, or Sepigel® 305 of the Seppic company; polyvinyl alcohols that are marketed, for example, under the commercial names Elvanol® of DuPont or Vinol® 523/540 of the Air Products company, and polyethylene glycol/polypropylene glycol copolymers that are marketed, for example, under the commercial designations Ucon® of Union Carbide.

Suitable natural film-forming polymers are, for example, cellulose derivatives, for example hydroxypropyl cellulose having a molecular weight from 30,000 to 50,000 g/mol, which is marketed for example under the commercial name Nisso SI® by the Lehmann & Voss company, Hamburg.

Setting polymers contribute to the hold, and/or to buildup of the hair volume and hair fullness, of the overall hairstyle. These so-called “setting” polymers are at the same time also film-forming polymers and are therefore generally typical substances for shape-imparting hair-treatment agents such as hair setting agents, hair foams, hair waxes, hair sprays. It is certainly possible for film formation to be localized, and for only a few fibers to be connected to one another.

Substances that furthermore impart hydrophobic properties to the hair are preferred in this context, since they decrease the hair's tendency to absorb humidity, i.e. water. This decreases loose hanging of strands of hair, and thus ensures long-term hairstyle construction and retention. The so-called “curl retention” test is often used as a test method for this. These polymeric substances can furthermore be incorporated successfully into leave-on and rinse-off hair therapies or shampoos. Because polymers are often multifunctional, i.e. exhibit multiple effects that are desired in terms of applications engineering, numerous polymers belong to several groups categorized in terms of their respective action, for example including in the CTFA handbook. Because of the specific significance of the setting polymers, these will therefore be listed explicitly in the form of their INCI names. This list therefore, of course, also specifically contains the aforementioned film-forming polymers.

Examples of usual film-forming setting polymers are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Amino ethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate.

Compositions according to the present invention contain by preference at least one film-forming and/or setting polymer that is selected from vinylpyrrolidone/vinyl acetate copolymers, vinyl acetate/crotonic acid copolymers, vinylcaprolactam/vinylpyrrolidone/dimethylaminoethyl methacrylate copolymers, octyl acrylamide/acrylate/butylaminoethyl methacrylate copolymers, and quaternized vinylpyrrolidone/dimethylaminoethyl methacrylate copolymers.

The film-forming and/or setting polymer is particularly preferably the vinylpyrrolidone/vinyl acetate copolymers Luviskol® VA 37 or PVP/VA Copolymer 60/40 W NP, the vinyl acetate/crotonic acid copolymer that is marketed under the commercial name Aristoflex® A 60, the vinylcaprolactam/vinylpyrrolidone/dimethylaminoethyl methacrylate copolymer having the commercial name Advantage® LC-E, the amphoteric octyl acrylamide/acrylate/butylaminoethyl methacrylate copolymer obtainable under the name Amphomer®, or the quaternized vinylpyrrolidone/dimethylaminoethyl methacrylate copolymer, quaternized with diethyl sulfate, that is marketed under the commercial name Gafquat® 755N.

Particularly preferably, compositions according to the present invention additionally contain at least one vinylpyrrolidone/vinyl acetate copolymer.

In summary, agents preferred according to the present invention are those in which the film-forming polymer and/or polymer having a setting effect on the hair is selected from at least one polymer of the group that is constituted from nonionic polymers based on ethylenically unsaturated monomers, nonionic cellulose derivatives, starch and derivatives thereof, chitosan and derivatives of chitosan, cationic cellulose derivatives, cationic copolymers of 3-(C₁ to C₆) alkyl-1-vinylimidazolinium, homopolymers and copolymers of diallyldimethylammonium, homopolymers and copolymers containing the structural unit of formula (M-1)

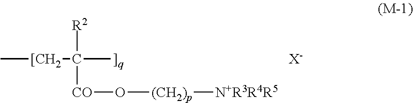

in which R²=—H or is —CH₃, R³, R⁴, and R⁵ are selected mutually independently from (C₁ to C₄) alkyl, (C₁ to C₄) alkenyl, or (C₂ to C₄) hydroxyalkyl groups, p=1, 2, 3, or 4, q is a natural number, and X⁻ is a physiologically acceptable organic or inorganic anion, amphoteric copolymers of N—(C₆ to C₁₀) alkyl acrylamide, amphoteric copolymers of N—(C₆ to C₁₀) alkyl methacrylamide, anionic polymers that comprise carboxylate and/or sulfonate groups, anionic polyurethanes.

As a third essential ingredient, the agents according to the present invention contain 0.5 to 30 wt %, by preference 1 to 25 wt %, more preferably 2.5 to 20 wt %, and in particular 5 to 15 wt % of at least one wax having a melting point in a range from 40° C. to 90° C. In general waxes have a solid to brittle hard consistency, coarse to finely crystalline, transparent to opaque but not glass-like, and melt above 40° C. without decomposition. Only slightly above the melting point their viscosity is already low, and they exhibit a highly temperature-dependent consistency and solubility. Natural vegetable waxes, for example, are preferred according to the present invention, for example candelilla wax, carnauba wax, Japan wax, sugar cane wax, ouricury wax, cork wax, sunflower wax, fruit waxes such as orange waxes, lemon waxes, grapefruit wax, and animal waxes, e.g. beeswax, shellac wax, and spermaceti. It can be particularly preferred for purposes of the invention to use hydrogenated or hardened waxes. Also usable as a wax component are chemically modified waxes, in particular the hard waxes such as e.g. montan ester waxes, hydrogenated jojoba waxes, and sasol waxes. Included among the synthetic waxes that are likewise preferred according to the present invention are, for example, polyalkylene waxes, in particular polyethylene waxes; and polyethylene glycol waxes, C₂₀ to C₄₀ dialkyl esters of dimer acids, C₃₀₋₅₀ alkyl beeswax, and alkyl and aryl esters of dimer fatty acids.

A particularly preferred wax component is selected from at least one ester of a saturated monovalent C₁₆ to C₅₀ alcohol and a saturated C₈ to C₃₆ monocarboxylic acid. Also included thereamong according to the present invention are lactides, the cyclic double esters of α-hydroxycarboxylic acids of the corresponding chain length. Esters of fatty acids and long-chain alcohols have proven particularly advantageous for the composition according to the present invention. The esters are made up of saturated, branched or unbranched monocarboxylic acids and saturated, branched or unbranched monovalent alcohols. Esters of aromatic carboxylic acids and/or hydroxycarboxylic acids (e.g. 12-hydroxystearic acid) and saturated, branched or unbranched alcohols are also usable according to the present invention, provided the wax component has a melting point >50° C.

It is particularly preferred to select the wax components from the group of esters of saturated, branched or unbranched alkanecarboxylic acids having a chain length from 12 to 24 carbon atoms, and from saturated, branched or unbranched alcohols having a chain length from 16 to 50 carbon atoms, which have a melting point >50° C.

C₁₆₋₃₆ alkyl stearates and C₁₈₋₃₈ alkylhydroxystearoyl stearates, C₂₀₋₄₀ alkyl erucates, and cetearyl behenate, can be particularly preferred as a wax component. The wax or the wax components have a melting point >50° C., preferably >60° C.

A particularly preferred embodiment of the invention contains as a wax component a C₂₀ to C₄₀ alkyl stearate. This ester is known by the names Kesterwachs® K82H or Kesterwachs® K80H, and is marketed by Koster Keunen Inc. This is a synthetic imitation of the monoester fraction of beeswax, and is notable for its hardness, oil-gelling capability, and wide compatibility with lipid components. A further particularly preferred embodiment of the invention contains as a wax component cetearyl behenate, i.e. mixtures of cetyl behenate and stearyl behenate. This ester is known by the name Kesterwachs® K62 and is marketed by Koster Keunen Inc.

Further preferred wax components having a melting point >50° C. are triglycerides of saturated and optionally hydroxylated C₁₂₋₃₀ fatty acids, such as hardened triglyceride fats (hydrogenated palm oil, hydrogenated coconut oil, hydrogenated castor oil), glyceryl tribehenate (Tribehenin), or glyceryl tri-12-hydroxystearate, furthermore synthetic full esters of fatty acids and glycols or polyols having 2 to 6 carbon atoms, provided they have a melting point above 50° C., for example preferably C₁₈-C₃₆ Acid Triglyceride (Syncrowax® HGL-C). Hydrogenated castor oil, obtainable e.g. as a commercial product Cutina® HR, is particularly preferred according to the present invention as a wax component.

Further preferred lipid components or wax components having a melting point >50° C. are the saturated linear C₁₄ to C₃₆ carboxylic acids, in particular myristic acid, palmitic acid, stearic acid, and behenic acid, as well as mixtures of these compounds, e.g. Syncrowax® AW IC (C₁₈ to C₃₆ fatty acids) or Cutina® FS 45 (palmitic and stearic acid).

Further preferred lipid components or wax components having a melting point in the range from 30 to 150° C. are linear, saturated C₈₋₃₀ fatty acids. Linear, saturated C₁₀₋₂₂ fatty acids are preferred. Preferred fatty acids are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, eleostearic acid, arachidonic acid, gadoleic acid, behenic acid, and erucic acid, as well as industrial mixtures thereof. The use of stearic acid is particularly preferred. The fatty acids used can carry one or more hydroxy groups. Preferred examples thereof are α-hydroxy-C₈ to C₁₈ carboxylic acids as well as 12-hydroxystearic acid.

Further preferred lipid components or wax components having a melting point in the range from 30 to 150° C. are fatty alcohols. Fatty alcohols that can be used are saturated, unbranched fatty alcohols having 6 to 30, preferably 10 to 22, and very particularly 12 to 22 carbon atoms. Decanol, octanol, erucyl alcohol, ricinol alcohol, 12-hydroxystearyl alcohol, stearyl alcohol, cetyl alcohol, lauryl alcohol, myristyl alcohol, arachidyl alcohol, capryl alcohol, caprinyl alcohol, and behenyl alcohol are usable, for example, for purposes of the invention.

Preferred compositions according to the present invention are characterized in that the wax component is selected from esters of a saturated monovalent C₁₆ to C₆₀ alkanol and a saturated C₈ to C₃₆ monocarboxylic acid, in particular cetyl behenate, stearyl behenate, and C₂₀ to C₄₀ alkyl stearate, glycerol triesters of saturated linear C₁₂ to C₃₀ carboxylic acids, which can be hydroxylated, candelilla wax, carnauba wax, beeswax, saturated linear C₁₄ to C₃₆ carboxylic acids, as well as mixtures of the substances recited above. Particularly preferred wax component mixtures are selected from mixtures of cetyl behenate, stearyl behenate, hardened castor oil, palmitic acid, and stearic acid. Further particularly preferred wax component mixtures are selected from mixtures of C₂₀ to C₄₀ alkyl stearate, hardened castor oil, palmitic acid, and stearic acid.

Particularly preferred compositions according to the present invention are characterized in that the wax component is selected from mixtures of esters of a saturated monovalent C₁₆ to C₆₀ alkanol and a saturated C₈ to C₃₆ monocarboxylic acid, in particular C₂₀ to C₄₀ alkyl stearate, glycerol triesters of saturated linear C₁₂ to C₃₀ carboxylic acids, which can be hydroxylated, in particular hydrogenated castor oil, and saturated linear C₁₄ to C₃₆ carboxylic acids, in particular palmitic acid and stearic acid.

In summary, agents particularly preferred according to the present invention are characterized in that they contain at least one wax from the group of beeswax, carnauba wax, candelilla wax, montan wax, cetyl palmitate, or mixtures thereof.

The agents according to the present invention contain the ingredients in a cosmetically acceptable carrier.

Preferred cosmetically acceptable carriers are aqueous, alcoholic, or aqueous alcoholic media having by preference at least 10 wt % water, based on the entire agent. The alcohols contained can be, in particular, the lower alcohols having 1 to 4 carbon atoms usually used for cosmetic purposes, for example ethanol and isopropanol.

Organic solvents or a mixture of solvents having a boiling point below 400° C. can be contained as additional co-solvents, in a quantity from 0.1 to 15 weight percent, preferably from 1 to 10 weight percent, based on the total agent. Unbranched or branched hydrocarbons such as pentane, hexane, isopentane, and cyclic hydrocarbons such as cyclopentane and cyclohexane, are particularly suitable as additional co-solvents. Further particularly preferred water-soluble solvents are glycerol, ethylene glycol, butylene glycol, and propylene glycol, in a quantity of up to 30 wt % based on the weight of the entire agent.

Very particularly preferred agents according to the present invention have a high concentration of water. It has been found that shine, remodulability, and degree of hold can be established particularly successfully with the compositions according to the present invention if they contain large proportions of water. Particularly preferred agents according to the present invention are therefore characterized in that they contain, based on their weight, 40 to 95 wt %, by preference 45 to 92.5 wt %, more preferably 50 to 90 wt %, even more preferably 55 to 87.5 wt %, and in particular 60 to 85 wt % water.

The applicability of the compositions can be further enhanced by the use of small quantities of one or more polyvalent alcohols. Preferred agents according to the present invention contain, based on their weight, 0.25 to 5 wt %, by preference 0.5 to 4 wt %, more preferably 0.75 to 3 wt %, and in particular 1 to 2.5 wt % of at least one polyvalent alcohol from the group of glycerol and/or 1,2-propanediol.

The agents preferably have a pH from 2 to 11. Particularly preferably, the pH range is between 4 and 9. Unless otherwise noted the indications as to pH refer, for purposes of this document, to the pH at 25° C.

The agents according to the present invention can furthermore contain the adjuvants and additives that are usually added to the respective cosmetic agents.

Care-providing substances are to be recited in particular as suitable adjuvants and additives. These are utilized in both skin treatment agents and hair treatment agents, and with appropriate selection of the care-providing substance can be incorporated, for example, into creams, shampoos, hair rinses, hair therapies, gels, pump and aerosol sprays, and foam products.

An agent according to the present invention can contain as a care-providing substance, for example, at least one protein hydrolysate and/or one of its derivatives.

Protein hydrolysates are product mixtures obtained by the acid-, base-, or enzyme-catalyzed breakdown of proteins. The molecular weight of the protein hydrolysates usable according to the present invention is between 75 (the molecular weight of glycine) and 200,000; the molecular weight is equal to preferably 75 to 50,000 dalton, and very particularly preferably to 75 to 20,000 dalton.

Protein hydrolysates of both vegetable as well as animal or marine or synthetic origin can be used according to the present invention. Animal protein hydrolysates are, for example, protein hydrolysates of elastin, collagen, keratin, silk, and milk protein, which can also be present in the form of salts. Such products are marketed, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), Sericin (Pentapharm), and Kerasol® (Croda). The use of silk protein hydrolysates is of particular interest. The protein hydrolysates are contained in the agents according to the present invention, for example, in concentrations from 0.01 wt % up to 20 wt %, by preference from 0.05 wt % up to 15 wt %, and very particularly preferably in quantities from 0.05 wt % up to 5 wt %, based in each case on the total utilization preparation.

Cationic surfactants are further suitable as a care-providing substance of a different class of compound. Cationic surfactants of the quaternary ammonium compound, esterquat, and amidoamine types are preferred according to the present invention. Preferred quaternary ammonium compounds are ammonium halides, in particular chlorides and bromides, such as alkyltrimethylammonium chlorides, dialkyldimethylammonium chlorides, and trialkylmethylammonium chlorides, for example cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride, as well as the imidazolium compounds known by the INCI names Quaternium-27 and Quaternium-83. The long alkyl chains of the aforementioned surfactants preferably have 10 to 18 carbon atoms. Esterquats are known substances that contain both at least one ester function and at least one quaternary ammonium group as a structural element. Preferred esterquats are quaternized ester salts of fatty acids with triethanolamine, quaternized ester salts of fatty acids with diethanolalkylamines, and quaternized ester salts of fatty acids with 1,2-dihydroxypropyldialkylamines. Such products are marketed, for example, under the trademarks Stepantex®, Dehyquart®, and Armocare®. Examples of such esterquats are the products Armocare® VGH-70—an N,N-bis(2-palmitoyloxyethyl)dimethylammonium chloride—as well as Dehyquart® F-75, Dehyquart® C-4046, Dehyquart® L80, and Dehyquart® AU-35. The alkylamidoamines are usually manufactured by amidation of natural or synthetic fatty acids and fatty acid cuts with dialkylaminoamines. One compound from this group of substances that is particularly suitable according to the present invention is represented by the stearamidopropyldimethylamine available commercially under the designation Tegoamid® S 18.

The cationic surfactants are contained in the agents according to the present invention preferably in quantities from 0.05 to 10 wt %, based on the total utilization preparation. Quantities from 0.1 to 5 wt % are particularly preferred.

Care-providing polymers are likewise suitable as a care-providing substance. Be it noted at this juncture that some care-providing polymers also exhibit film-forming and/or setting properties, and can therefore also be recited when listing suitable film-forming and/or setting polymers.

Cationic polymers are a first group of care-providing polymers. “Cationic” polymers are to be understood as polymers that comprise in the main chain and/or side chain a group that can be “temporarily” or “permanently” cationic. According to the present invention, those polymers that comprise a cationic group regardless of the pH of the agent are referred to as “permanently cationic.” These are, as a rule, polymers that contain a quaternary nitrogen atom, for example in the form of an ammonium group. Preferred cationic groups are quaternary ammonium groups. Those polymers in which the quaternary ammonium group is bonded via a C₁₋₄ hydrocarbon group to a main polymer chain constructed from acrylic acid, methacrylic acid, or derivatives thereof, have proven particularly suitable.

Homopolymers of the general formula (G1-I)

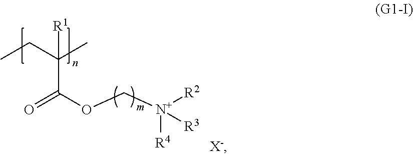

in which R¹=—H or is —CH₃, R², R³, and R⁴ are selected mutually independently from C₁₋₄ alkyl, alkenyl, or hydroxyalkyl groups, m=1, 2, 3, or 4, n is a natural number, and X⁻ is a physiologically acceptable organic or inorganic anion, as well as copolymers made up substantially of the monomer units presented in formula (G1-I) as well as nonionogenic monomer units, are particularly preferred cationic polymers. In the context of these polymers, the ones preferred according to the present invention are those for which at least one of the following conditions is valid: R¹ denotes a methyl group, R², R³, and R⁴ denote methyl groups, m has the value of 2.

Halide ions, sulfate ions, phosphate ions, methosulfate ions, as well as organic ions such as lactate, citrate, tartrate, and acetate ions are appropriate, for example, as physiologically acceptable counter ions X⁻. Halide ions, in particular chloride, are preferred.

A particularly suitable homopolymer is the poly(methacryloyloxyethyltrimethylammonium) chloride (crosslinked, if desired) having the INCI name Polyquaternium-37. The crosslinking can be accomplished, if desired, with the aid of olefinically polyunsaturated compounds, for example divinylbenzene, tetraallyloxyethane, methylene bisacrylamide, diallyl ether, polyallylpolyglyceryl ether, or allyl ethers of sugars or sugar derivatives such as erythritol, pentaerythritol, arabitol, mannitol, sorbitol, sucrose, or glucose. Methylene bisacrylamide is a preferred crosslinking agent.

The homopolymer is preferably used in the form of a nonaqueous polymer dispersion that should comprise a polymer proportion not less than 30 wt %. Such polymer dispersions are obtainable commercially under the designations Salcare® SC 95 (approx. 50% polymer proportion, further components: mineral oil (INCI name: Mineral Oil) and tridecylpolyoxypropylenepolyoxyethylene ether (INCI name: PPG-1-Trideceth-6)), and Salcare® SC 96 (approx. 50% polymer proportion, further components: mixture of diesters of propylene glycol with a mixture of caprylic and capric acid (INCI name: Propylene Glycol Dicaprylate/Dicaprate) and tridecylpolyoxypropylenepolyoxyethylene ether (INCI name: PPG-1-Trideceth-6)).

Copolymers having monomer units according to formula (G1-I) preferably contain acrylamide, methacrylamide, acrylic acid C₁₋₄ alkyl esters, and methacrylic acid C₁₋₄ alkyl esters as nonionogenic monomer units. Among these nonionogenic monomers, the acrylamide is particularly preferred. These copolymers as well, as in the case of the homopolymers described above, can be crosslinked. A copolymer preferred according to the present invention is the crosslinked acrylamide-methacryloyloxyethyltrimethylammonium chloride copolymer. Such copolymers, in which the monomers are present at a weight ratio of approximately 20:80, are obtainable commercially as an approximately 50% nonaqueous polymer dispersion under the name Salcare® SC 92.

Further preferred cationic polymers are, for example,
quaternized cellulose derivatives such as those commercially obtainable under the designations Celquat® and Polymer JR®. The compounds Celquat® H 100, Celquat® L 200, and Polymer JR® 400 are preferred quaternized cellulose derivatives,
cationic alkylpolyglycosides according to German patent DE 44 13 686,
cationized honey, for example the commercial product Honeyquat® 50,
cationic guar derivatives, such as in particular the products marketed under the trade names Cosmedia® Guar and Jaguar®,
polysiloxanes having quaternary groups, for example the commercially obtainable products Q2-7224 (manufacturer: Dow Corning; a stabilized trimethylsilylamodimethicone), Dow Corning® 929 Emulsion (containing a hydroxylamino-modified silicone that is also referred to as Amodimethicone), SM-2059 (manufacturer: General Electric), SLM-55067 (manufacturer: Wacker), as well as Abil-Quat 3270 and 3272 (manufacturer: Th. Goldschmidt; diquaternary polydimethylsiloxanes, Quaternium-80).
polymeric dimethyldiallylammonium salts and copolymers thereof with esters and amides of acrylic acid and methacrylic acid. The products obtainable commercially under the designations Merquat® 100 (poly(dimethyldiallylammonium chloride)) and Merquat® 550 (dimethyldiallylammonium chloride/acrylamide copolymer) are examples of such cationic polymers,
quaternized poly(vinylalcohol),
and the polymers known under the names Polyquaternium-2, Polyquaternium-17, Polyquaternium-18, and Polyquaternium-27, having quaternary nitrogen atoms in the main polymer chain.

Further cationic polymers usable according to the present invention are the so-called “temporarily cationic” polymers. These polymers usually contain an amino group that at specific pH values exists as a quaternary ammonium group (and is thus cationic). Chitosan and derivatives thereof are, for example, preferred, for example those readily available commercially under the commercial designations Hydagen® CMF, Hydagen® HCMF, Kytamer® PC, and Chitolam® NB/101.

The agents according to the present invention contain the care-providing, cationic, and/or amphoteric polymers preferably in a quantity from 0.01 to 5 wt %, in particular in a quantity from 0.1 to 2 wt %, based in each case on the total utilization preparation.

Agents according to the present invention that are further preferred are characterized in that they additionally contain (a) care-providing substance(s) in quantities, based on their weight, from 0.001 to 10 wt %, by preference 0.005 to 7.5 wt %, particularly preferably 0.01 to 5 wt %, and in particular 0.05 to 2.5 wt %, (a) preferred care-providing substance(s) being selected from the group: L-carnitine and/or salts thereof; panthenol and/or pantothenic acid; the 2-furanones and/or derivatives thereof (in particular pantolactone); taurine and/or salts thereof; niacinamide; ubiquinone; ectoin; allantoin.

L-carnitine (IUPAC name: (R)-(3-carboxy-2-hydroxypropyl)-N,N,N-trimethylammonium hydroxide) is a naturally occurring vitamin-like substance. L-carnitine derivatives preferred according to the present invention are selected in particular from acetyl-L-carnitine, L-carnitine fumarate, L-carnitine citrate, lauroyl-L-carnitine, and particularly preferably L-carnitine tartrate. The aforesaid L-carnitine compounds are obtainable, for example, from Lonza GmbH (Wuppertal, Germany). Agents preferred according to the present invention are characterized in that they contain, based on their weight, 0.001 to 10 wt %, by preference 0.005 to 7.5 wt %, particularly preferably 0.01 to 5 wt %, and in particular 0.05 to 2.5 wt % L-carnitine or L-carnitine derivatives, where preferred L-carnitine derivatives are selected from acetyl-L-carnitine, L-carnitine fumarate, L-carnitine citrate, lauroyl-L-carnitine, and in particular L-carnitine tartrate.

Panthenol (IUPAC name: (+)-(R)-2,4-dihydroxy-N-(3-hydroxypropyl)-3,3-dimethylbutyramide) is converted in the body to pantothenic acid. Pantothenic acid is a vitamin from the group of the B vitamins (vitamin B5) Preferred agents according to the present invention are characterized in that they contain, based on its weight, 0.01 to 5 wt %, by preference 0.05 to 2.5 wt %, particularly preferably 0.1 to 1.5 wt %, and in particular 0.25 to 1 wt % panthenol ((±)-2,4-dihydroxy-N-(3-hydroxypropyl)-3,3-dimethylbutyramide).

A further care enhancer that is preferred for use, which possesses activating properties, is taurine. Agents preferred according to the present invention contain, based on their weight, 0.01 to 15 wt %, by preference 0.025 to 12.5 wt %, particularly preferably 0.05 to 10 wt %, more preferably 0.1 to 7.5 wt %, and in particular 0.5 to 5 wt % taurine (2-aminoethanesulfonic acid).

A further preferred group of care enhancers in the agents according to the present invention is vitamins, provitamins, or vitamin precursors. These are described below:

In summary, agents according to the present invention that contain, based on their weight, 0.1 to 5 wt %, by preference 0.2 to 4 wt %, particularly preferably 0.25 to 3.5 wt %, more preferably 0.5 to 3 wt %, and in particular 0.5 to 2.5 wt % vitamins and/or provitamins and/or vitamin precursors that by preference are assigned to the groups A, B, C, E, F, and H, where preferred agents contain-2,4-dihydroxy-N-(3-hydroxypropyl)-3,3-dimethylbutyramide (provitamin B₅) and/or pantothenic acid (vitamin B₃, vitamin B₅) and/or niacin, niacinamide or nicotinamide (vitamin B₃) and/or L-ascorbic acid (vitamin C) and/or thiamin (vitamin B₁) and/or riboflavin (vitamin B₂, vitamin G) and/or biotin (vitamin B₇, vitamin H) and/or folic acid (vitamin B₉, vitamin B_{c} or vitamin M) and/or vitamin B₆ and/or vitamin B₁₂, are preferred.

It has been found that certain quinones possess a particular suitability as a care enhancer. Particularly preferred agents according to the present invention are characterized in that they contain as a care-providing substance, based on their weight, 0.0001 to 5 wt %, preferably 0.001 to 0.5 wt %, and particularly preferably 0.005 to 0.1 wt % of at least one ubiquinone and/or at least one ubiquinol and/or at least one derivative of said substances, where preferred agents contain a ubiquinone of formula (Ubi)

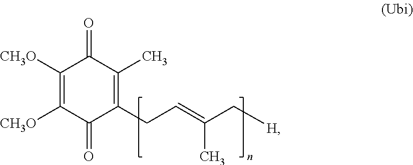

in which n denotes the values 6, 7, 8, 9, or 10, particularly preferably 10 (coenzyme Q10).

As a further care enhancer, the agents according to the present invention can contain ectoin. Ectoin ((4S)-2-methyl-1,4,5,6-tetrahydropyrimidine-4-carboxylic acid) is a natural substance belonging to the group of the compatible solutes.

As a further constituent, the agents according to the present invention can contain at least one carbohydrate from the group of the monosaccharides, disaccharides, and/or oligosaccharides. Hair treatment agents preferred according to the present invention are characterized here in that they contain as a care-providing substance, based on their weight, 0.01 to 5 wt %, by preference 0.05 to 4.5 wt %, particularly preferably 0.1 to 4 wt %, more preferably 0.5 to 3.5 wt %, and in particular 0.75 to 2.5 wt % carbohydrate(s) selected from monosaccharides, disaccharides, and/or oligosaccharides, preferred carbohydrates being selected from
monosaccharides, in particular D-ribose and/or D-xylose and/or L-arabinose and/or D-glucose and/or D-mannose and/or D-galactose and/or D-fructose and/or sorbose and/or L-fucose and/or L-rhamnose,
disaccharides, in particular sucrose and/or maltose and/or lactose and/or trehalose and/or cellobiose and/or gentobiose and/or isomaltose.

In a further preferred embodiment, the agents according to the present invention can contain emulsifier agents (F). Emulsifier agents cause the formation, at the phase interface, of water- and/or oil-stable adsorption layers that prevent the dispersed droplets from coalescing, and thereby stabilize the emulsion. Emulsifier agents are therefore, like surfactants, constructed from a hydrophobic and a hydrophilic molecule part. Hydrophilic emulsifier agents preferentially form o/w emulsions, and hydrophobic emulsifier agents preferentially form w/o emulsions. An “emulsion” is to be understood as a droplet-like distribution (dispersion) of one liquid in another liquid, with the expenditure of energy to create stabilizing phase interfaces by means of surfactants. Selection of these emulsifying surfactants or emulsifier agents is based on the substances to be dispersed and the respective external phase, and on the fineness of the emulsion particles. Emulsifier agents usable according to the present invention are, for example:
addition products of 4 to 30 mol ethylene oxide and/or 0 to 5 mol propylene oxide with linear fatty alcohols having 8 to 22 carbon atoms, with fatty acids having 12 to 22 carbon atoms, and with alkylphenols having 8 to 15 carbon atoms in the alkyl group;
C₁₂ to C₂₂ fatty acid mono- and diesters of addition products of 1 to 30 mol ethylene oxide with polyols having 3 to 6 carbon atoms, in particular with glycerol;
addition products of ethylene oxide and polyglycerol with methyl glucoside fatty acid esters, fatty acid alkanolamides, and fatty acid glucamides;
C₈ to C₂₂ alkyl mono- and oligoglycosides and ethoxylated analogs thereof, where degrees of oligomerization from 1.1 to 5, in particular 1.2 to 2.0, and glucose as the sugar component, are preferred;
mixtures of alkyl(oligo)glucosides and fatty alcohols, for example the commercially available product Montanov® 68;
addition products of 5 to 60 mol ethylene oxide with castor oil and hardened castor oil;
partial esters of polyols having 3 to 6 carbon atoms with saturated fatty acids having 8 to 22 carbon atoms;
Sterols. “Sterols” are understood as a group of steroids that carry a hydroxyl group on the third carbon atom of the steroid structure and are isolated both from animal tissue (zoosterols) and from vegetable fats (phytosterols). Examples of zoosterols are cholesterol and lanosterol. Examples of suitable phytosterols are ergosterol, stigmasterol, and sitosterol. Sterols called “mycosterols” are also isolated from fungi and yeasts.
Phospholipids. These are understood as principally the glucose phospholipids, which are obtained e.g. as lecithins and/or phosphatidylcholines from, for example, egg yolk or plant seeds (e.g. soybeans).
fatty acid esters of sugars and sugar alcohols, such as sorbitol,
polyglycerols and polyglycerol derivatives such as, for example, polyglycerol poly-12-hydroxystearate (commercial product Dehymuls® PGPH),
linear and branched fatty acids having 8 to 30 carbon atoms, and the sodium, potassium, ammonium, calcium, magnesium, and zinc salts thereof.

The agents according to the present invention contain the emulsifier agents preferably in quantities from 0.1 to 25 wt %, in particular 0.5 to 15 wt %, based on the entire agent. The compositions according to the present invention can preferably contain at least one nonionogenic emulsifier agent having an HLB value from 8 to 18. Nonionogenic emulsifier agents having an HLB value from 10 to 15 can be particularly preferred according to the present invention.

Depending on the nature of the agent according to the present invention, it can be necessary for it additionally to contain at least one surfactant. This applies in particular to skin cleaning agents and shampoos. Other agents as well, however, for example hair rinses, hair therapies, and specific styling agents, in particular styling foams, can also contain surfactants.

Cationic surfactants, for example, such as those already described above a suitable care-proving substances, can be used. With regard to the preferred cationic surfactants and the quantities used, the statements made above apply accordingly.

Besides or instead of the cationic surfactants, the agents can contain further surfactants or emulsifier agents; in principle, both anionic as well as ampholytic and nonionic surfactants, and all types of known emulsifier agents, are suitable. The group of the ampholytic or also amphoteric surfactants encompasses zwitterionic surfactants and ampholytes. The surfactants can already have an emulsifying effect.

All anionic surface-active substances suitable for use on the human body are, in principle, appropriate as anionic surfactants. These are characterized by an anionic group imparting water solubility, for example a carboxylate, sulfate, sulfonate, or phosphate group, and a lipophilic alkyl group having approximately 8 to 30 carbon atoms. Glycol ether or polyglycol ether groups, ester, ether, and amide groups, and hydroxyl groups can additionally be contained in the molecule. Examples of suitable anionic surfactants are, in each case in the form of the sodium, potassium, and ammonium salts and the mono-, di, and trialkanolammonium salts having 2 to 4 carbon atoms in the alkanol group:
linear and branched fatty acids having 8 to 30 carbon atoms (soaps),
ethercarboxylic acids of the formula R—O—(CH₂—CH₂O)ₓ—CH₂—COOH, in which R is a linear alkyl group having 8 to 30 carbon atoms and x=0 or is 1 to 16,
acyl sarcosides having 8 to 24 carbon atoms in the acyl group,
acyl taurides having 8 to 24 carbon atoms in the acyl group,
acyl isethionates having 8 to 24 carbon atoms in the acyl group,
sulfosuccinic acid mono- and dialkyl esters having 8 to 24 carbon atoms in the alkyl group, and sulfosuccinic acid monoalkylpolyoxyethyl esters having 8 to 24 carbon atoms in the alkyl group and 1 to 6 oxyethyl groups,
linear alkanesulfonates having 8 to 24 carbon atoms,
linear alpha-olefinsulfonates having 8 to 24 carbon atoms,
alpha-sulfo fatty acid methyl esters of fatty acids having 8 to 30 carbon atoms,
alkyl sulfates and alkyl polyglycol ether sulfates of the formula R—O(CH₂—CH₂—O)ₓ—OSO₃H, in which R is a preferably linear alkyl group having 8 to 30 carbon atoms and x=0 or is 1 to 12,
mixtures of surface-active hydroxysulfonates,
sulfated hydroxyalkylpolyethylene glycol ethers and/or hydroxyalkylenepropylene glycol ethers,
sulfonates of unsaturated fatty acids having 8 to 24 carbon atoms and 1 to 6 double bonds,
esters of tartaric acid and citric acid with alcohols representing addition products of approximately 2 to 15 molecules of ethylene oxide and/or propylene oxide with fatty alcohols having 8 to 22 carbon atoms,
alkyl and/or alkenyl ether phosphates,
sulfated fatty acid alkylene glycol esters of formula (E-1-II)

R⁷CO(AlkO)ₙSO₃M (E-1-II)
in which R⁷CO denotes a linear or branched, aliphatic, saturated and/or unsaturated acyl residue having 6 to 22 carbon atoms, Alk denotes CH₂CH₂, CHCH₃CH₂, and/or CH₂CHCH₃, n denotes numbers from 0.5 to 5, and M denotes a cation, as described in German Application 197 36 906,
amide ethercarboxylic acids,
condensation products of C₃ to C₃₀ fatty alcohols with protein hydrolysates and/or amino acids and derivatives thereof, known to one skilled in the art as protein fatty acid condensates, such as, for example, the Lamepon® grades, Gluadin® grades, Hostapon® KCG, or the Amisoft® grades.

Preferred anionic surfactants are alkyl sulfates, alkyl polyglycol ether sulfates, and ethercarboxylic acids having 10 to 18 carbon atoms in the alkyl group and up to 12 glycol ether groups in the molecule, sulfosuccinic acid mono- and dialkyl esters having 8 to 18 carbon atoms in the alkyl group, and sulfosuccinic acid monoalkylpolyoxyethyl esters having 8 to 18 carbon atoms in the alkyl group and 1 to 6 oxyethyl groups, monoglycerol disulfates, alkyl and alkenyl ether phosphates, as well as protein fatty acid condensates.

“Zwitterionic surfactants” refers to those surface-active compounds that carry in the molecule at least one quaternary ammonium group and at least one —COO⁽⁻⁾ or —SO₃⁽⁻⁾ group. Particularly suitable zwitterionic surfactants are the so-called betaines, such as N-alkyl-N,N-dimethylammonium glycinates, for example cocalkyldimethylammonium glycinate, N-acylaminopropyl-N,N-dimethylammonium glycinates, for example cocacylaminopropyldimethylammonium glycinate, and 2-alkyl-3-carboxymethyl-3-hydroxyethylimidazolines, having in each case 8 to 18 carbon atoms in the alkyl or acyl group, as well as cocacylaminoethylhydroxyethylcarboxymethyl glycinate. A preferred zwitterionic surfactant is the fatty acid amide derivative known by the INCI name Cocamidopropyl Betaine.

“Ampholytes” are understood to be those surface-active compounds that contain in the molecule, besides a C₈ to C₂₄ alkyl or acyl group, at least one free amino group and at least one —COOH or —SO₃H group, and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids, having in each case approximately 8 to 24 carbon atoms in the alkyl group. Particularly preferred ampholytes are N-cocalkylaminopropionate, cocacylaminoethylaminopropionate, and C₁₂ to C₁₈ acyl sarcosine.

Nonionic surfactants contain as a hydrophilic group, for example, a polyol group, a polyalkylene glycol ether group, or a combination of a polyol ether and polyglycol ether group. Such compounds are, for example:
addition products of 2 to 50 mol ethylene oxide and/or 1 to 5 mol propylene oxide with linear and branched fatty alcohols having 8 to 30 carbon atoms, with fatty acids having 8 to 30 carbon atoms, and with alkylphenols having 8 to 15 carbon atoms in the alkyl group,
addition products, end-capped with a methyl or C₂ to C₆ alkyl residue, of 2 to 50 mol ethylene oxide and/or 1 to 5 mol propylene oxide with linear and branched fatty alcohols having 8 to 30 carbon atoms, with fatty acids having 8 to 30 carbon atoms, and with alkylphenols having 8 to 15 carbon atoms in the alkyl group, such as, for example, the grades obtainable under the marketing designations Dehydrol® LS, Dehydrol® LT (Cognis),
C₁₂ to C₃₀ fatty acid mono- and diesters of addition products of 1 to 30 mol ethylene oxide with glycerol,
addition products of 5 to 60 mol ethylene oxide with castor oil and hardened castor oil,
polyol fatty acid esters such as, for example, the commercial product Hydagen® HSP (Cognis), or Sovermol® grades (Cognis),
alkoxylated triglycerides,
alkoxylated fatty acid alkyl esters of formula (E4-I)

R¹CO—(OCH₂CHR²)_{w}OR³ (E4-I),
in which R¹CO denotes a linear or branched, saturated and/or unsaturated acyl residue having 6 to 22 carbon atoms, R² denotes hydrogen or methyl, R³ denotes linear or branched alkyl residues having 1 to 4 carbon atoms, and w denotes numbers from 1 to 20,
amine oxides,
sorbitan fatty acid esters and addition products of ethylene oxide with sorbitan fatty acid esters, for example the polysorbates,
sugar fatty acid esters and addition products of ethylene oxide with sugar fatty acid esters,
addition products of ethylene oxide with fatty acid alkanolamides and fatty amines,
sugar surfactants of the alkyl and alkenyl oligoglycoside types, in accordance with formula (E4-II)

R⁴O-[G]ₚ (E4-II),

in which R⁴ denotes an alkyl or alkenyl residue having 4 to 22 carbon atoms, G denotes a sugar residue having 5 or 6 carbon atoms, and p denotes numbers from 1 to 10. They can be obtained in accordance with relevant methods of preparative organic chemistry. The preferred alkyl and/or alkenyl oligoglycosides are alkyl and/or alkenyl oligoglucosides. The index number p in the general formula (E4-II) indicates the degree of oligomerization (DP), i.e. the distribution of mono- and oligoglycosides, and denotes a number between 1 and 10. The alkyl or alkenyl residue R⁴ can derive from primary alcohols having 4 to 11, by preference 8 to 10 carbon atoms. Alkyl oligoglucosides based on hardened C_{12/14} coconut alcohol having a DP of 1 to 3 are preferred.

Alkylene oxide addition products with saturated linear fatty alcohols and fatty acids, having respectively 2 to 30 mol ethylene oxide per mol fatty alcohol and/or fatty acid, have proven to be preferred nonionic surfactants. Preparations having outstanding properties are likewise obtained if they contain, as nonionic surfactants, fatty acid esters of ethoxylated glycerol. These compounds are characterized by the following parameters: The alkyl residue R contains 6 to 22 carbon atoms and can be both linear and branched. Primary linear aliphatic residues, and aliphatic residues methyl-branched in the 2-position, are preferred. Such alkyl residues are, for example, 1-octyl, 1-decyl, 1-lauryl, 1-myristyl, 1-cetyl, and 1-stearyl. 1-Octyl, 1-decyl, 1-lauryl, and 1-myristyl are particularly preferred. When so-called “oxo alcohols” are used as the initial materials, compounds having an odd number of carbon atoms in the alkyl chain predominate.

The compounds having alkyl groups used as surfactants can in each case be uniform substances. It is preferred as a rule, however, to proceed from natural vegetable or animal raw materials when producing these substances, so that substance mixtures having different alkyl chain lengths, dependent on the particular material, are obtained.

The further surfactants are used as a rule in quantities from 0.1 to 45 wt %, preferably 0.5 to 30 wt %, and very particularly preferably from 0.5 to 25 wt %, based on the respective total composition. The quantity used depends substantially on the purpose served by the agent according to the present invention. If it is a shampoo or another cleaning agent, surfactant quantities even above 45 wt % are usual.

The agents according to the present invention can be formulated in all forms usual for cosmetic agents, for example in the form of solutions that can be applied onto the skin or hair as face lotions or hair lotions or a pump or aerosol spray, in the form of creams, emulsions, waxes, gels, or also surfactant-containing foaming solutions, or other preparations that are suitable for use on the skin or hair.

Very particularly preferred agents according to the present invention are characterized in that they involve a styling gel, a styling cream, a styling wax, a pump hair spray, an aerosol hair spray, a pump hair foam, an aerosol hair foam, or combinations thereof.

The agents according to the present invention are, however, by preference agents for the temporary deformation of keratinic fibers, i.e. styling agents. Preferred styling agents are styling gels, pump hair sprays, aerosol hair spray, pump hair foams, and aerosol hair foams.

In the context of the present application, “styling gels” is the general term for clear or opaque products, styling waxes, styling creams, styling lotions, styling jellies, etc. This term ultimately encompasses all agents for the styling of hair that are not hair sprays or foams.

“Hair foams” are understood in this context as compositions that form a foam upon removal from a suitable container. It can be necessary to add to the agents ingredients that promote foam formation or stabilize a foam once it has formed. Surfactants and/or emulsifier agents, as already described above, are particularly suitable for this. Surfactants from the group of the cationic surfactants are preferably used.

Hair creams and hair gels as a rule contain structuring agents and/or thickening polymers which serve to impart the desired consistency to the products. Structuring agents and/or thickening polymers are used typically in a quantity from 0.1 to 10 wt %, based on the entire product. Quantities from 0.5 to 5 wt %, in particular 0.5 to 3 wt %, are preferred. Because the polymer combination used according to the present invention has self-thickening properties, however, the addition of further structuring agents and/or thickening polymers is not absolutely necessary. The agents according to the present invention by preference contain no further structuring agents and/or thickening polymers.

If the agents according to the present invention involve an aerosol product, the latter obligatorily contains a propellant. Propellants suitable according to the present invention are, for example, N₂O, dimethyl ether, CO₂, air, and alkanes having 3 to 5 carbon atoms such as propane, n-butane, isobutane, n-pentane, and isopentane, and mixtures thereof. Dimethyl ether, propane, n-butane, isobutane, and mixtures thereof are preferred. The aforesaid alkanes, mixtures of the aforesaid alkanes, or mixtures of the aforesaid alkanes with dimethyl ether are preferably used as the only propellant. The invention also, however, expressly encompasses the concurrent use of propellants of the fluorochlorocarbon type, but in particular the fluorocarbons. For a given spray apparatus, the sizes of the aerosol droplets and/or of the foam bubbles, and the respective size distribution, can be adjusted by way of the quantitative ratio of propellant to the remaining constituents of the preparations.

The quantity of propellant used varies as a function of the specific composition of the agent, the packaging used, and the desired type of product (e.g. hair spray or hair foam). When conventional spray apparatuses are used, aerosol foam products contain the propellant preferably in quantities from 1 to 35 wt % based on the total product. Quantities from 2 to 30 wt %, in particular from 3 to 15 wt %, are particularly preferred. Aerosol sprays generally contain larger quantities of propellant. In this case the propellant is used preferably in a quantity from 30 to 98 wt % based on the total product. Quantities from 40 to 95 wt %, in particular from 50 to 95 wt %, are particularly preferred.

The aerosol products can be manufactured in usual fashion. As a rule all the constituents of the respective agent, with the exception of the propellant, are introduced into a suitable pressure-tight container. The latter is then sealed with a valve. Lastly, the desired quantity of propellant is introduced using conventional techniques.

A second subject of the invention is therefore a method for the temporary deformation of keratinic fibers in which the cosmetic agent according to the present invention is applied onto the hair as a pump hair spray, aerosol hair spray, pump hair foam, aerosol hair foam, styling gel, styling cream, styling wax, or combinations thereof, and is optionally worked into the hair using the palms of the hands and/or the fingers.

The statements made regarding the agents according to the present invention apply mutatis mutandis to the method according to the present invention. The desired deformation of the hair can be brought about using the fingers or hands, and with suitable conventional aids such as a comb or brush.

A third subject of the present invention is the use of the agents according to the present invention for the temporary deformation of keratinic fibers. The agents according to the present invention, and products that contain said agents, are notable in particular for the fact that they impart a very strong hairstyle hold to hair treated with them, without thereby making the hair brittle or inflexible. A pleasant, soft feel is instead achieved.

The agents according to the present invention can be formulated in any form usual for cosmetic agents, for example in the form of solutions that can be applied onto the skin or hair as a face lotion or hair lotion or as a pump or aerosol spray, in the form of creams, emulsions, waxes, gels, or also surfactant-containing foaming solutions or other preparations that are suitable for application to the skin or hair.

### EXAMPLES

The following quantitative indications are to be understood, unless otherwise noted, as percentages by weight.

Styling agents E1 to E4 according to the present invention were produced in accordance with Table 1 below.

TABLE 1

| Raw material (INCI name) | E1 | E2 | E3 | E4 |
|---|---|---|---|---|
| Simulgel EPG¹ | 0.5 | 0.6 | 0.7 | 1.0 |
| PVP/VA Copolymer 60/40 | 1.0 | 1.0 | 1.25 | 1.5 |
| Beeswax | 3.0 | 3.5 | 4.0 | 4.5 |
| Carnauba wax | 3.0 | 3.0 | 3.0 | 3.0 |
| C₁₈ fatty alcohol ethoxylate with | 2.0 | 2.0 | 2.0 | 2.0 |
| 21 EO | | | | |
| PEG-7 Glycerol Cocoate | 3.0 | 3.0 | 3.0 | 3.0 |
| Cetyl/stearyl alcohol | 2.0 | 2.0 | 2.0 | 2.0 |
| Stearyl alcohol | 1.0 | 1.0 | 1.0 | 1.0 |
| C₁₂₋₁₅ alkylbenzoate | 3.0 | 3.0 | 3.0 | 3.0 |
| Glycerol | 1.0 | 1.0 | 1.0 | 1.0 |
| 1,2-Propanediol | 1.0 | 1.0 | 1.0 | 1.0 |
| Phenoxyethanol | 0.5 | 0.5 | 0.5 | 0.5 |
| Dye | 0.002 | 0.002 | 0.002 | 0.002 |
| Perfume | 0.10 | 0.10 | 0.10 | 0.20 |
| Ethanol 96%, denatured | — | — | — | 10.70 |
| Water, deionized | to 100 | to 100 | to 100 | to 100 |

¹INCI name: Sodium Acrylate/Sodium Acryloyldimethyltaurate Copolymer, Polyisobutene, Caprylyl/Capryl Glucoside (Seppic)

The agents were produced by mixing, in usual fashion, the raw materials recited in the table, and were used by rubbing them onto the palms of the hands followed by application onto dry or wet hair for hairstyling.

The hair had a smooth shine, outstanding hairstyle hold, and outstanding remodulability.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic agent comprising, in a cosmetically acceptable carrier, based in each case on its weight:
a) 0.01 to 3 wt % of at least one copolymer A that includes at least one structural unit according to formula (I) and at least one structural unit according to formula (II) 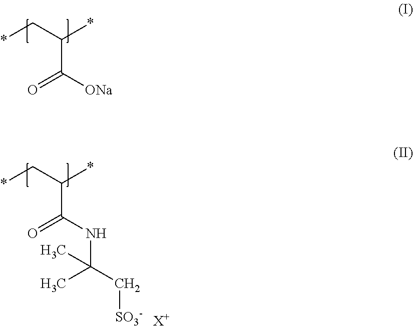
in which X⁺ denotes a physiologically acceptable cation,
b) 0.05 to 10 wt % of at least one film-forming polymer, and
c) 0.5 to 30 wt % of at least one wax having a melting point in a range from 40° C. to 90° C.

2. The agent according to claim 1, wherein the at least one copolymer A has molar mass from 10 to 750 kDa.

3. The agent according to claim 1, wherein the agent includes, based on its weight as a ready-to-use agent, 0.025 to 2.5 wt % copolymer(s) A.

4. The agent according to claim 1, wherein the film-forming polymer is selected from at least one polymer of the group consisting of nonionic polymers based on ethylenically unsaturated monomers, nonionic cellulose derivatives, starch and derivatives thereof, chitosan and derivatives of chitosan, cationic cellulose derivatives, cationic copolymers of 3-(C₁ to C₆) alkyl-1-vinylimidazolinium, homopolymers and copolymers of diallyldimethylammonium, homopolymers and copolymers containing the structural unit of formula (M-1) 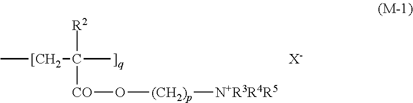
wherein R²=—H or is —CH₃, R³, R⁴, and R⁵ are selected mutually independently from (C₁ to C₄) alkyl, (C₁ to C₄) alkenyl, or (C₂ to C₄) hydroxyalkyl groups, p=1, 2, 3, or 4, q is a natural number, and X⁻ is a physiologically acceptable organic or inorganic anion, amphoteric copolymers of N—(C₆ to C₁₀) alkyl acrylamide, amphoteric copolymers of N—(C₆ to C₁₀) alkyl methacrylamide, anionic polymers that comprise carboxylate and/or sulfonate groups, and anionic polyurethanes.

5. The agent according to claim 1, wherein the at least one wax is selected from the group consisting of beeswax, carnauba wax, candelilla wax, montan wax, cetyl palmitate, and mixtures thereof.

6. The agent according to claim 1, further comprising, based on its weight, 40 to 95 wt % water.

7. The agent according to claim 1, further comprising, based on its weight, 0.25 to 5 wt % at least one polyvalent alcohol from the group consisting of glycerol and/or 1,2-propanediol.

8. The agent according to claim 1, wherein the agent is at least one agent selected from the group consisting of styling gel, a styling cream, a styling wax, a pump hair spray, an aerosol hair spray, a pump hair foam, and an aerosol hair foam.

9. A method for the temporary deformation of keratinic fibers, comprising:
applying the cosmetic agent according to claim 1 onto the hair as one or more of a pump hair spray, aerosol hair spray, pump hair foam, aerosol hair foam, styling gel, styling cream, and styling wax; and
optionally working the agent into the hair using the palms of the hands and/or the fingers.
